(19) United States
(12) Patent Application Publication
(10) Pub. No.: US 2005/0010150 A1
(43) Pub. Date: Jan. 13, 2005

(54) Customizable orthotic device with adjustable support and forces

(76) Inventor: Igal Firsov, Kiryat Ono (IL)

Correspondence Address: Mark Friedman;Bill Polkinghorn, Discovery Dispatch, 9003 Florin Way, Upper Marlboro, MD, 20772, US

(21) Appl. No.: 10/493,474

(22) PCT Filed: Oct. 28, 2002
(86) PCT No.: PCT/IL02/00862

Related U.S. Application Data
(60) Provisional application No. 60/330,695, filed on Oct. 29, 2001.

(51) Int. Cl. A61F 5/00

(52) U.S. Cl. 602/19

(57) ABSTRACT

A customizable orthotic bracing device with adjustable support and forces is disclosed. The device is to be applied to the body of an individual for treatment and rehabilitation of disorders of the musculoskeletal and nervous system. The device includes a wearable garment, and a plurality of support elements. The support elements are of variable and adjustable flexibility and are connected to the wearable garment.

### FIELD AND BACKGROUND OF THE INVENTION

The present invention relates to an orthotic device and, more particularly, to a customizable orthotic bracing device with adjustable support and forces for treatment and rehabilitation of various disorders of the musculoskeletal and nervous system which present with abnormalities of posture, muscle tone and motor function.

The list of disorders which present with abnormalities in muscle tone, posture, and motor function is long and includes such problems as cerebral palsy, scoliosis, motor pareses and paralyses (of various causes), dystonia, and injuries of the spine and joints, to name but a few. Such problems, even when the immediate site of pathology is localized, have far-flung consequences for the entire musculoskeletal and locomotor systems. Effective treatment and rehabilitation of such problems requires complex, global therapies that take into account and have an effect on the entire musculoskeletal and locomotor systems.

Cerebral palsy is a chronic and persistent disorder of movement and posture caused by non-progressive pathological processes of the immature central nervous system. Injury to the brain may occur before, during or shortly after birth. The overall incidence in Western countries of cerebral palsy is between 1.5 and 2.5 per 1000 live births. An estimated 500,000 to 700,000 children and adults in the United States manifest symptoms of cerebral palsy and about 9,000 new infants develop the disorder annually. Management of the child with cerebral palsy is a complex challenge.

Physiotherapy is the principal non-surgical form of treatment. Its aims are to prevent contractures and deformities and to promote functionally useful posture and movements. Cerebral palsy typically presents with spasticity of muscle that results in the limb being held in abnormal postures. Because muscle growth depends to a certain extent on muscle stretch, if abnormal postures are maintained for extended periods, muscle growth will be impaired. Eventually, the muscle will become short and contractures develop. Further, abnormal muscle movements tend to interfere with joint function. As a result affected children may develop deformities of the foot or of the hip joint (which may become dysplastic or eventually completely dislocate) for example. In general, such deformities can be minimized or prevented by splinting to maintain the joints and muscles in certain more natural, often neutral, positions. Further, the basic premise of physiotherapy is that disordered movements are improved by manipulation of and exercise with the affected muscles. Such treatments are based on the experiences of the past in treating children who suffered from infantile anterior poliomyelitis. The spastic child though stiff, is generally weak, and exercising spontaneously is difficult.

Movement and exercise of splinted muscles is also important to further prevent other complications arising from immobilization including metabolic disturbances (including bone demineralization), muscular atrophy, increased spasticity, and hypertension.

Thus it would be desirable to have devices which can be used for splinting and bracing various regions of the body which can be adjusted and customized so as to be usable in varying circumstances. Such variations include not only those of age and size, but also include variations in the regions of the body affected, as well as variations in the type of musculo-skeletal symptomatology (regardless of etiology) such as increased or decreased tone or the presence or absence of weakness or paralysis to name but a few. Such a device should also be adjustable, depending on varying circumstances, to be capable of fixing, stabilizing and immobilizing regions of the body including the musculo-skeletal apparatus, removing loads, applying forces to manipulate the region in various vectors with variable intensity, and be suitable for attachment of external devices to promote movement of a region. It should be able to perform one function on one region and another in a different region, and be capable of adjustment in real-time. For example, it should be able to fix one region while causing manipulation of another and allowing supported movement of still another.

There exists in the prior art numerous examples of splints, braces and supports, but none that meet the above requirements. For example, U.S. Pat. No. 5,613,941 to Prengler discloses a joint support apparatus for wrapping around a joint to protect and support the joint. Such an apparatus removes loads and supports the joint while allowing motion, but without either immobilizing the joint firmly or actively imparting forces to move the joint. It is applicable to varying individual joints but is not an integrated device for use simultaneously on multiple affected regions of the body. Further while it may stabilize a single injured, weakened or affected joint, it does not act upon remote regions involved in a pathological condition, directly or indirectly contributing to the symptoms of the problem. U.S. Pat. No. 6,142,965 to Mathewson discloses a brace constructed from flexible material designed to restrict rotational movement about a joint, which provides active resistance to rotation. Such a device is applicable only to a limb and is used to prevent injury of limb joints and not actively correct postural problems and those involving the trunk and spine and other regions. Devices such as those disclosed in U.S. Pat. No. 5,967,998 to Modglin and U.S. Pat. No. 6,213,968 to Heinz et al are orthotic braces which support the torso and spine (although only the lumbo-sacral and not the thoracic and cervical), and are adjustable, however the adjustments are only to allow the brace to conform to the torso even with changes in position of the wearer and not to permit the application of manipulating forces. They are only for stabilization, and do not allow limitation of movement for example only in specific directions. They do not allow specific localization of restriction of movement either: for example, allowing rotation only to the right at the level of L2 without restricting movement at other levels. The inflatable wearable traction device disclosed in U.S. Pat. No. 5,950,628 to Dunfee suffers from similar limitations.

U.S. Pat. No. 6,213,922 to Afanasenko et al. is directed to a device for treatment of patients with disturbed posture and motor activity comprising shoulder, pelvic, knee, pedal, elbow, hand, and finger supports, all of them interconnected by fixing elements, which are shaped as elastic tie-members and placed on the surface of the patient's body in antagonistic pairs. Each tie member is connected to two supports (through a lock) and includes a tension adjuster. The device suffers from a number of limitations however. The point of attachment of the fixing element to the support is not adjustable according to the particular circumstances of treatment. The fixing elements are only attached to two supports and can not be attached to more than two supports. The tension adjustment is not made in real time. Vertical spinal support (a “backbone”) capable of being adjusted to modify the force and direction of support is not provided. Full rigid support of the entire body is not possible. The device uses only single supports at each region with only few fixing elements limiting the directions and specificity of support and manipulation available. The creation of support points at multiple different levels within a single region (e.g. trunk, chest and spine, or forearm) is not possible. Each support is separately applied making application complex. There is no provision for attachment for additional control units or other devices to actively move or ambulate the patient.

There is thus a widely recognized need for, and it would be highly advantageous to have, a customizable orthotic bracing device with adjustable support, forces and pressure points for treatment and rehabilitation of various disorders of the musculoskeletal and nervous system which present with abnormalities of posture, muscle tone and motor function, devoid of the above limitations.

### SUMMARY OF THE INVENTION

The present invention successfully addresses the shortcomings of the presently known configurations by providing a customizable orthotic bracing device with adjustable support and forces for treatment and rehabilitation of various disorders of the musculo-skeletal and nervous system which present with abnormalities of posture, muscle tone and motor function.

According to one aspect of the present invention there is provided a customizable orthotic bracing device with adjustable support and forces, the device to be applied to the body of an individual, for treatment and rehabilitation of disorders of the musculoskeletal and nervous system, the device including a wearable garment, and a plurality of support elements, the support elements being of variable and adjustable flexibility, the support elements being connected to the wearable garment.

According to further features in preferred embodiments of the invention described below, the orthotic bracing device further includes at least one axial spanning element, the at least one axial spanning element being of variable and adjustable flexibility, the at least one axial spanning element being connected to at least two of the support elements.

According to additional features in the described preferred embodiments the support elements are contained within layers of the garment.

According to still additional features in the described preferred embodiment the support elements are located on the outer surface of the garment.

According to still further features in the described preferred embodiments the garment covers substantially the entire body of the individual.

According to still further features in the described preferred embodiments the garment is composed of a plurality of individual compartments.

According to still further features in the described preferred embodiments the compartments enclose a plurality of spaces, wherein at least one insert is inserted into at least one of the spaces.

According to still further features in the described preferred embodiments the at least one insert is rigid.

According to still further features in the described preferred embodiments the at least one insert is buoyant.

According to still further features in the described preferred embodiments the at least one insert is fabricated from a polystyrene foam.

According to still further features in the described preferred embodiments the garment is fabricated of a waterproof material.

According to still further features in the described preferred embodiments the device is adapted for use in hydrotherapy.

According to still further features in the described preferred embodiments the plurality of support elements are arranged in a parallel configuration on at least one region of the body of the individual.

According to still further features in the described preferred embodiments at least one of the support elements is constructed from a plurality of support segments.

According to still further features in the described preferred embodiments the support elements are arranged horizontally.

According to still further features in the described preferred embodiments at least one of the support elements is connected to a sternal fastener for providing additional rigidity analogous to a sternal bone of the individual.

According to still further features in the described preferred embodiments at least one of the support elements is connected to a spinal fastener for providing additional rigidity analogous to a vertebral bone of the individual.

According to still further features in the described preferred embodiments the orthotic bracing device includes a plurality of spinal fasteners.

According to still further features in the described preferred embodiments the device includes a plurality of parallel support elements, each of the support elements being connected to a spinal fastener, which are axially connected in a posterior portion of the device so as to constitute a backbone.

According to still further features in the described preferred embodiments at least two adjacent of the spinal fasteners are connected by at least one tension connector.

According to still further features in the described preferred embodiments the flexibility of the at least one tension connector is variable.

According to still further features in the described preferred embodiments the flexibility of the at least one tension connector is adjustable.

According to still further features in the described preferred embodiments the orthotic bracing device further includes at least one moveable joint connection functionally connected to at least one support element.

According to still further features in the described preferred embodiments the orthotic bracing device further includes at least one moveable joint correction functionally connected to at least one axial spanning element.

According to still further features in the described preferred embodiments the orthotic bracing device father includes a positional support element for assisting the individual to maintain a desired posture, the standing support element functionally connected to at least one of the support elements.

According to still further features in the described preferred embodiments the desired posture is standing.

According to still further features in the described preferred embodiments the desired posture is sitting.

According to still further features in the described preferred embodiments the positional support element is connected to the at least one of the support elements by at least one ball and socket joint connection.

According to still further features in the described preferred embodiments at least one ball of the at least one ball and socket joint is a component of the positional support element.

According to still further features in the described preferred embodiments the at least one ball is connected to the positional support element by at least one element conferring a variable degree of freedom of movement.

According to still further features in the described preferred embodiments the at least one ball is connected to the positional support element by at least one element conferring an adjustable degree of freedom of movement.

According to still further features in the described preferred embodiments the positional support element includes a standing support bar functionally connected to the at least one support element.

According to still further features in the described preferred embodiments the standing support bar is functionally connected to the at least one support element by a movable joint connection.

According to still further features in the described preferred embodiments the standing support bar is of adjustable length.

According to still further features in the described preferred embodiments the standing support bar includes at least one hinge joint along the length of the standing support bar.

According to still further features in the described preferred embodiments the positional support element includes a walker frame, the walker frame functionally connected to the support element by a vertical rod.

According to still further features in the described preferred embodiments the walker frame is connected to the vertical rod by a frame connection allowing adjustment of a position of the vertical rod vertically and in a forward and backward direction relative to the walker frame.

According to still further features in the described preferred embodiments the walker frame is further connected to at least one wheel.

According to still further features in the described preferred embodiments the walker frame is further connected to two wheels.

According to still further features in the described preferred embodiments the walker frame is further connected to three wheels.

According to still further features in the described preferred embodiments the walker frame is further connected to four wheels.

According to still further features in the described preferred embodiments the orthotic bracing device further includes a brake mechanism attached to the at least one wheel.

According to still further features in the described preferred embodiments the positional support element is adapted so as to be foldable.

According to still further features in the described preferred embodiments the positional support element includes a seat.

According to still further features in the described preferred embodiments the positional support element is adapted so as to allow the patient to move between standing and sitting positions.

According to still further features in the described preferred embodiments the orthotic racing device further includes a motion element for assisting the individual in moping at east one part of the body, the motion element functionally connected to at least one of the support elements.

According to still further features in the described preferred embodiments the orthotic bracing device further includes a motor assembly functionally connected to at least one of the support elements for actively moving at least one portion of the body.

According to still further features in the described preferred embodiments the axial spanning element is a cable, adapted so as to capable of being tension adjustable, so as to adjust a degree of flexibility of the cable.

According to still further features in the described preferred embodiments the support elements are inflatable tubes.

According to still further features in the described preferred embodiments at least one of the support elements include a cable, adapted so as to capable of being tension adjustable, so as to adjust a degree of flexibility of the cable, thereby adjusting the flexibility of the support element.

According to still further features in the described preferred embodiments the device is adapted for use in treatment of cerebral palsy.

According to still further features in the described preferred embodiments the device is adapted for use in treatment of scoliosis.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a front view of a preferred embodiment of the device of the present invention;

FIG. 2 is a side view of a preferred embodiment of the device of the present invention;

FIG. 3 is a back view of a preferred embodiment of the device of the present invention;

FIG. 4 is a front view of a preferred embodiment of the device of the present invention;

FIG. 5 is a front view of a preferred embodiment of the trunk portion of the device of the present invention;

FIG. 6 is a diagram of a preferred embodiment of the fastening element of the device of the present invention;

FIG. 7 is a back view of a preferred embodiment of the trunk portion of the device of the present invention;

FIG. 8 is a front view of a preferred embodiment of the device of the present invention;

FIG. 9 is a close up schematic diagram of elements of the device of the present invention;

FIG. 10 is a schematic diagram of a preferred embodiment of a lock fastener of the device of the present invention;

FIG. 11 is a schematic diagram of a preferred embodiment of the device of the present invention;

FIG. 12 is a perspective view schematic diagram of a preferred embodiment of a support element of the device according to the present invention;

FIG. 13 is a schematic diagram of a preferred embodiment of a connector according to the present invention;

FIG. 14 is a schematic diagram of a preferred embodiment of the device of the present invention;

FIG. 15 is a close-up schematic diagram of a preferred embodiment of the device of the present invention;

FIG. 16 is a perspective view schematic diagram of a preferred embodiment of the device of the present invention;

FIG. 17 is a frontal view schematic diagram of a preferred embodiment of the device of the present invention;

FIG. 18 is a close-up schematic diagram of a preferred embodiment of the device of the present invention;

FIG. 19 is a cut away view schematic diagram of a preferred embodiment of the device of the present invention;

FIG. 20 is a schematic diagram of a preferred embodiment of the device of the present invention illustrating foldability and portability;

FIG. 21 is a further schematic diagram of a preferred embodiment of the device of the present invention;

FIG. 22 is another schematic diagram of a preferred embodiment of the device of the present invention; and,

FIG. 23 is yet another schematic diagram of a preferred embodiment of the device according to the present invention.

### DESCRIPTION OF THE PREFERRED EMBODIMENTS

The present invention is of a customizable orthotic bracing device with adjustable support and forces which can be used for treatment and rehabilitation of various disorders of the musculoskeletal and nervous system which present with abnormalities of posture, muscle tone and motor function. Specifically, the present invention can be easily applied and adjusted and can be used to fix, support, and restrict motion of regions of the body of a patient, at different levels, as well as to apply forces to manipulate the regions of the body.

For purposes of this specification and accompanying claims, the term “patient” is used to refer to an individual who is suffering from some symptom or disorder, awaiting or under the care of a health care professional, and includes individuals who are suffering from some symptom or disorder even if not under the care of a health care professional.

The principles and operation of a customizable orthotic bracing device with adjustable support, forces and pressure points according to the present invention may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

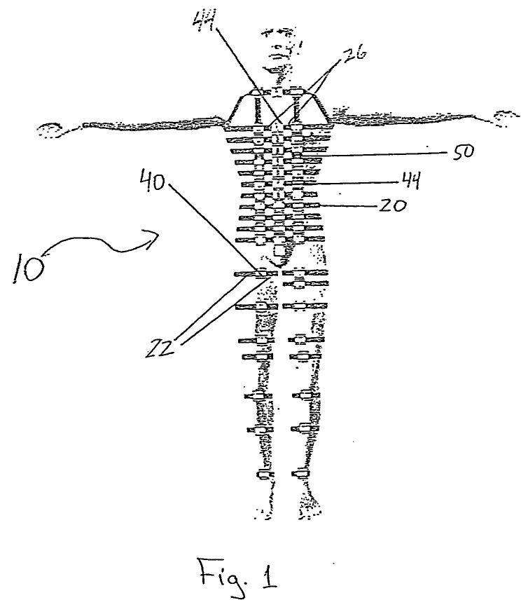

Referring now to the drawings, FIG. 1 illustrates the orthotic bracing device according to the present invention. Orthotic device 10 is essentially designed as an external skeleton. Device 10 provides both rigid support to various regions of the body of a patient while also generating forces of variable vector and intensity acting on the regions of the body so as to manipulate the regions. Such forces for example can apply traction to a region. Thus the generated forces can be conceived of as an external set of muscles which are used to function in the place of those that are not functioning properly in a disease state. Connections among the skeletal analogue elements act as joints. Thus device 10 actually acts as an external musculo-skeletal system.

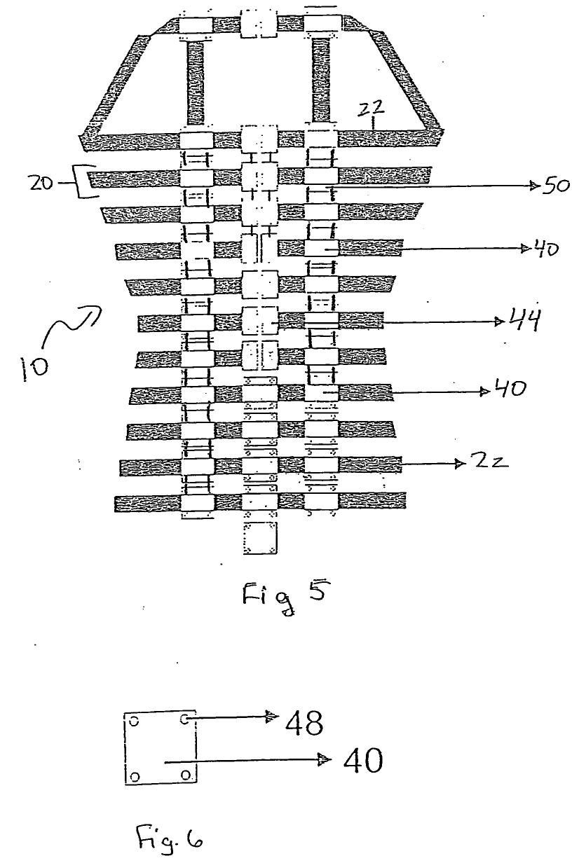

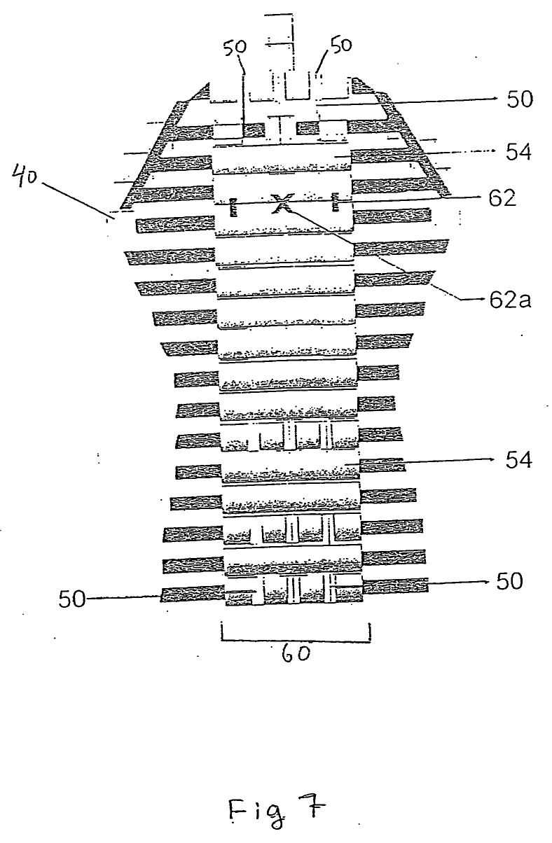

In some embodiments of device 10, device 10 covers all or most of the body (as in FIGS. 1-3), while in other preferred in embodiments the external skeleton is only partial and covers only a portion of the body (FIGS. 5 and 6, for example.) Like reference numerals refer to like parts throughout the figures of the drawing.

The basic structure of device 10 is built around a plurality of support elements 20. Support elements 20 have an adjustable degree of rigidity ranging from absolutely elastic to fully rigid. When fully rigid, support elements 20 support and immobilize the region of the body on which that element 20 is applied. When less than fully rigid, but rather elastic, element 20 instead allows movement and can even impart a force on the region in which it is applied, particularly when connected to a more rigid element.

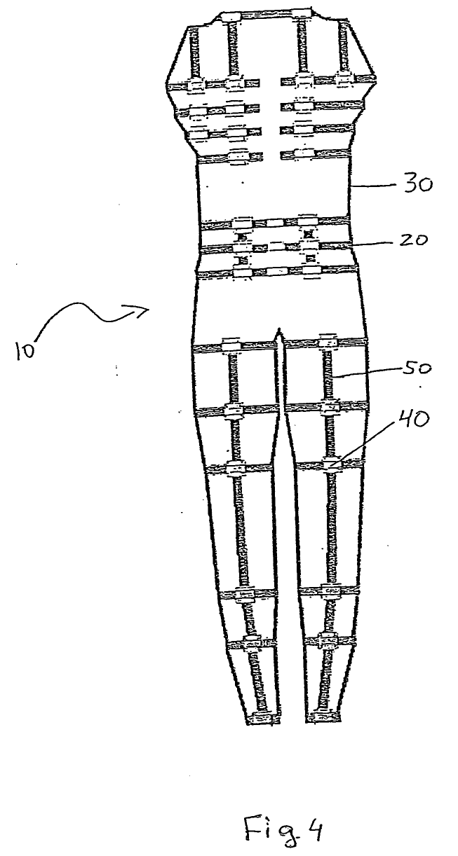

As seen best in FIG. 4, device 10 is preferably constructed in the form of a suit 30, designed to be worn on the body. Such a suit 30 is easily donned as a wearable garment, like a pair of overalls, simplifying application of device 10. Preferably suit 30 is constructed from a durable, non-allergenic, biocompatible fabric. Such a fabric (natural or synthetic) is preferably lightweight and does not overly interfere with the exchange of air, heat, moisture and the like between the surface of the body and the surrounding environment. The other elements of device 10 are contained within layers of suit 30 or in other embodiments are located on the outer surface of suit 30. Suit 30 may cover substantially the entire body (that is, except for the head, hands and feet, and in some cases, the neck) or some portion thereof. The area and position of covering is determined according to specific planned use of device 10. Suit 30 may be composed of separate pieces or a single piece which may utilize a closure such as zipper, snaps, or hooks and eyes as non-limiting examples for closure.

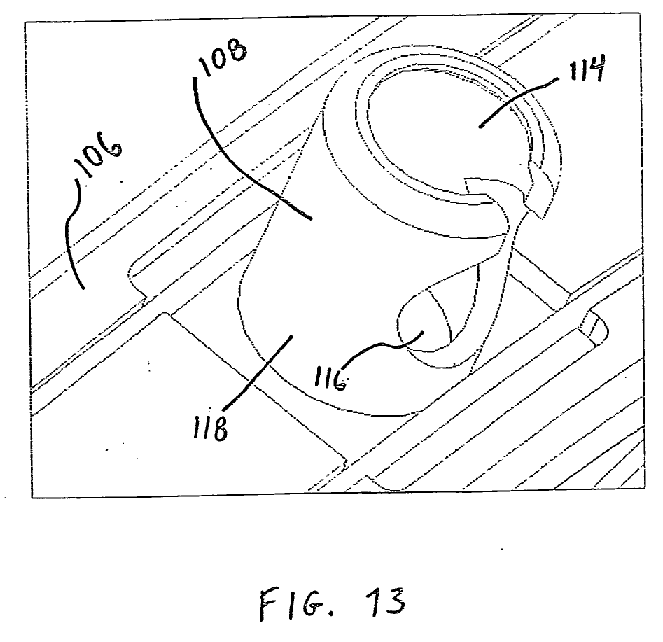

In certain preferred embodiments suit 30 is constructed in the form of a network of individual cells, pockets or compartments such that that the fabric enclosures of the cells enclose a plurality of spaces 102 into which various inserts 104 may be inserted, as shown in FIG. 12. Spaces 102 may be of varying size, shape, and density, and inserts 104 may be composed of varying materials to confer on suit 30 alternative properties. For example, inserts may be of a rigid material to enhance the support and rigidity of suit 30, for example along a certain axis depending on which cells are filled with this material. Alternatively an elastic material can be used for inserts 104 in other cells to allow a greater freedom of mobility along another axis. Additionally the fabric chosen for suit 30 itself can afford a degree of rigidity or flexibility to suit 30 and device 10.

For example, inserts 104 may be made of a buoyant material such as polystyrene or polyethylene foam or other materials such as are used in life vests and other flotation devices, so as to confer on suit 30 buoyancy. In certain preferred embodiments the buoyant material can be a gas as well as a solid material. This has application in the use of device 10 in hydrotherapy. In such a case, for example, the fabric of suit 30 is also composed of a waterproof material. This allows device 10 to be worn by the patient and used in a pool of water to reduce gravitational loading on the patient's musculoskeletal system (a major focus of the use of device 10 during the rehabilitation and treatment of diseases such as cerebral palsy), as well as for increased relaxation during therapeutic exercise regimens. The amount of buoyant material inserted can be varied to achieve the minimal necessary buoyancy of the patient. The buoyant inserts can also be selectively placed into certain cells occupying certain positions in suit 30 along certain axes and planes so as to adjust the positions of the body floating on and submerged in the water. Additional components such as external flotation elements or supporting components, including standing rods, and other support elements, as described hereinunder, can be attached to suit 30 to help maintain the patient in a desired position. For example, as illustrated below, attachment of a supporting component at the level of and in the field of the pelvis can help maintain the patient in a vertical position. Additional components and elements may be attached to suit 30 by any suitable form of attachment as is known in the art including, as non-limiting examples, clips, rings, hooks, and any other such fastener. Preferably such fasteners allow easy and rapid connection of additional components and elements.

Use of flotation can help enable placement of the patient in a new position by adjusting the water level or moving the patient along an inclined plane so as to submerge new segments and allow the patient to easily some to a new position in a gradual protected manner. The degree of immersion leaves portions of the trunk for example, above water level and the maintenance of vertical posture will be dependant on other factors such as other support mechanisms in device 10 above the water level or the patients own abilities to do so independent of device 10. After placement of the patient in the minimal necessary level of water various exercises can be performed directed to development of balance and other motor skills. The device can be connected further, as described hereinunder, to additional devices such as an underwater treadmill or other simulators to develop skills such as walking, as a non-limiting example. Use of buoyant inserts can also be used in adaptations of device 10 for use as a protective suit or for use for example in underwater rescue.

The plurality of support elements 20 are generally arranged in a generally parallel fashion on at least one region of the body. In various regions and in various configurations support elements 20 fully encircle the region circumferentially, while in other regions and in other configurations, support elements 20 encircle only a portion of the of the region but are connected to other elements. The construction of support elements 20 is further described hereinunder.

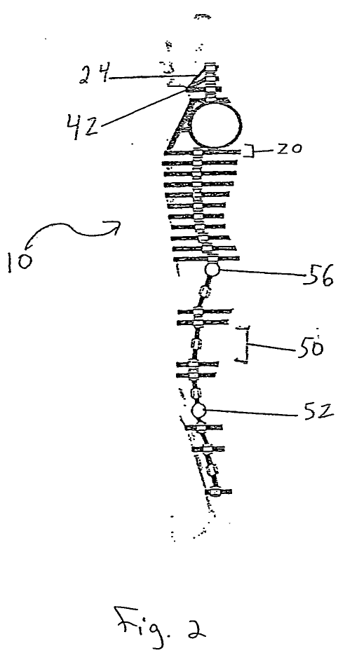

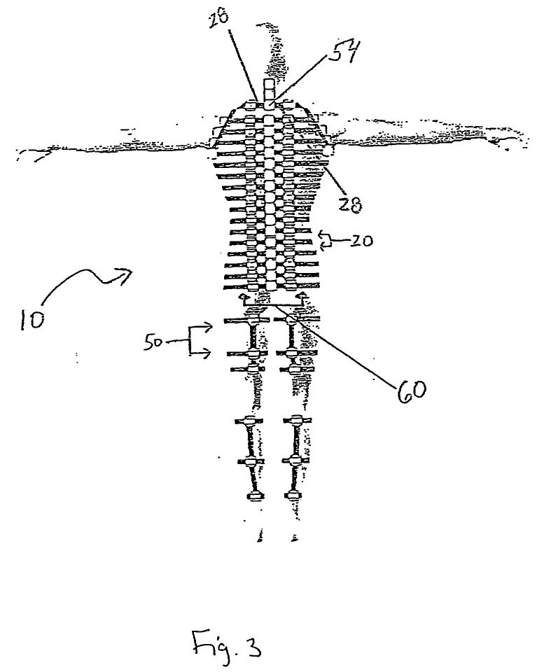

In a preferred embodiment of the present invention, as seen in FIG. 1, each of the support elements 20 includes more than one component support segment. Multiple support elements 20 are shown in FIG. 1 for example, where a specific support element 20 is composed of individual segments. Individual support segments are indicated by label 22. Each support segment 22 is connected to another component. A support segment 22 is connected to another support segment 22 through universal fastener 40 generally attached to a first end of each support segment 22. Other components may also be interposed between support elements 22 at the site of fastener 40. In certain uses, the second end of each support segment 22 is attached to another support segment 22, again through a universal fastener 40. In other uses and in other portions of device 10, a support segment 22 is connected to an axial spanning component 50 via connection of both to a universal fastener 40. Generally the support elements 20 run horizontally on the body and the axial spanning components 50 run vertically. Support elements 20 can also run at other angles as illustrated by identifying label 24 in FIG. 2. Note here as well that FIG. 2 shows 3 different support elements being connected together at a single universal fastener here specifically labeled as 42, illustrating that more than 2 support elements can be attached together. Certain support elements are attached to specialized fastening elements which perform additional functions. For example in FIG. 1, specific support segments 22 (which in this case are labeled 26 for identification) are attached to sternal fasteners 44. The at least one sternal fastener 44 generally located anteriorly, at the center of the torso, gives additional rigidity at the location of fastening and serves like the bones of the sternum. In a similar fashion, FIG. 3 illustrates that support elements over the back of the body may contain specific support segments (here identified as 28) which connect to at least one spinal fastener 54. Connection of support elements to spinal elements 54 or sternal fasteners 44 may be direct or via universal fastener 40. A plurality of spinal fasteners 54 runs vertically along the midline of the back analogous to the bodies of the vertebrae. At the back there are series of connections that axially (vertically) connect successive support elements 20 (including spinal fasteners 54) creating a backbone 60.

Adjacent layers of support elements surrounding the torso simulate a rib cage (FIG. 5). Sternal fasteners 44 and universal fasteners 40 of adjacent horizontal layers of support elements 20 (analogous to different ribs) in this region are connected amongst themselves in certain configurations by means of axial spanning components 50 of variable and adjustable elasticity to give either rigidity or allow movement at specific sites. Analogously various segments of backbone 60 are or are connected to further elements of variable and adjustable elasticity to confer rigidity or flexibility to the entire spinal component or to specific component regions. In FIG. 2 movable joint connections 52 and 56 are illustrated. Movable joint connection 52 is directly connected to an axial spanning element while movable joint connection 56 is attached via a universal fastener 40 to a support element 20. Movable joint connections allow freer movement in pre-determined planes at locations of joints. Construction of the joint connections allows adjustability of degree (angle) of movement and amount of force necessary to move the joint.

FIG. 6 illustrates a universal fastener 40 that has on it a plurality of sites of attachment 48 for connecting the various elements connected via fastener 40. Site of attachment 48 may be a hole or groove through which cables, screws, pins, wires, and the like may be fastened or pass through.

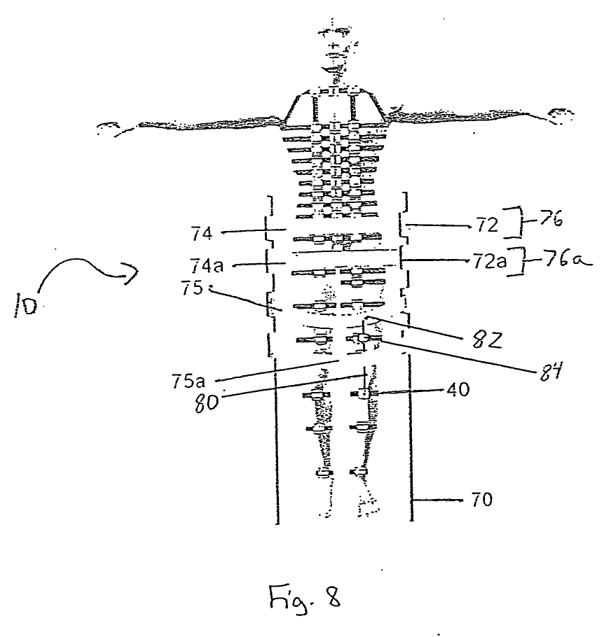

FIG. 7 illustrates a preferred embodiment of device 10 that covers the torso from a posterior view. A plurality of spinal fasteners 54 is seen within backbone 60. Axial spanning components 50 conferring varying degrees of rigidity and flexibility run vertically and connect adjacent spinal fasteners and support elements. Three sets of axial spanning components is illustrated in FIG. 7. Rigidity can be further enhanced through use of tension connectors 62 which attach adjacent spinal fasteners 54 either individually or in pairs (62a). Adjustable variable rigidity of connections allows creation of flexibility of movement or restriction of movement and can exert a force of traction and distraction. Forces and counter-forces can be applied. Axial support and rigidity allows for counterforces to be applied to overcome the forces of gravity and the imposed load on the musculo-skeletal system and structures.

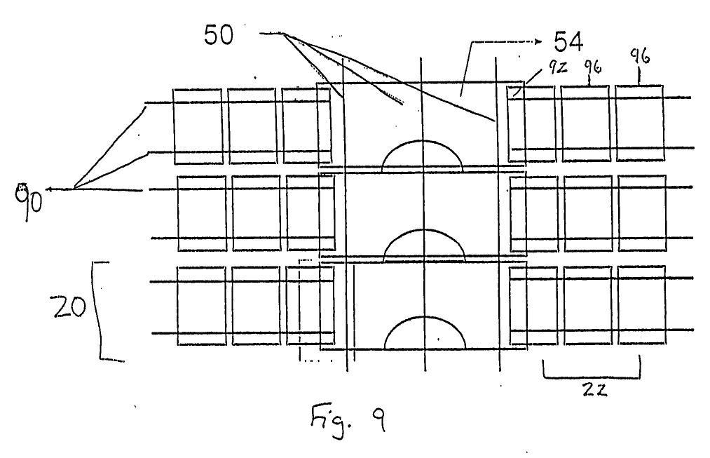

Creation of axial rigidity provides the opportunity for placing and maintaining the body in an upright posture. Device 10 through the use of universal fasteners permits the attachment of additional elements, such as devices for upright support and ambulation. Such an embodiment is illustrated in FIG. 8. At least one standing rod 70 (two are shown in FIG. 8) is connected to device 10 via universal fasteners 40. FIG. 8 illustrates a preferred embodiment in which such connection involves a cross complex 76 composed of rod connector 72 attached to a cross rod 74. Cross complex 76 is designed to be movable in a vertical direction from position 76 to 76a (components 72a, 742, and 76a are the same components, respectively as 72, 74 and 76, but moved to a different position) in order to alter the vertical gravitational loading on the legs and to help move the patient to a sitting position. In the embodiment illustrated in FIG. 8, a cabled cross complex 78 is also illustrated. Cabled complex 78 includes cabled cross connector 75. FIG. 8 illustrates that cabled complex 78 (and cabled cross connector 75) is movable in position in a vertical direction between the position indicated as 78 (and 75) and that indicated as 78a (and 75a). This can, for example be used to lift a leg. For such a use at least one cable 80 is fastened to at least one universal fastener 40 on the leg of the patient and is connected 82 to cabled cross connector 75. In certain embodiments a weight 84 is also attached to at least one cable 80 to add to the force of help use to move the leg.

In various embodiments other components can be added to the device. For example, wheels, with or without an axle can be added to attach to device 10 to enable it to function as a walker for independent walking. For example wheels could be added to the distal ends of standing rods 70. Further driving mechanisms, or motor assemblies, such as pistons, motors and other necessary connections and control mechanisms can be added to actively move portions of the body for physiotherapy or act as artificial muscles such as to aid in ambulation. Such a device for example could be used to perform repetitive stepping motions or treadmill like walking movements. In certain embodiments further devices such as a gyroscope are added to assist in maintaining balance.

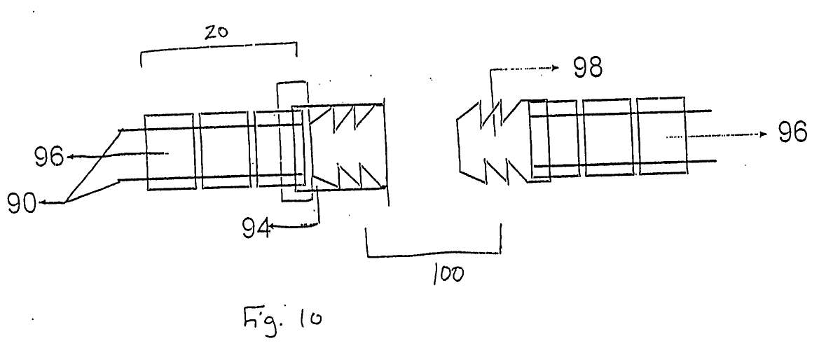

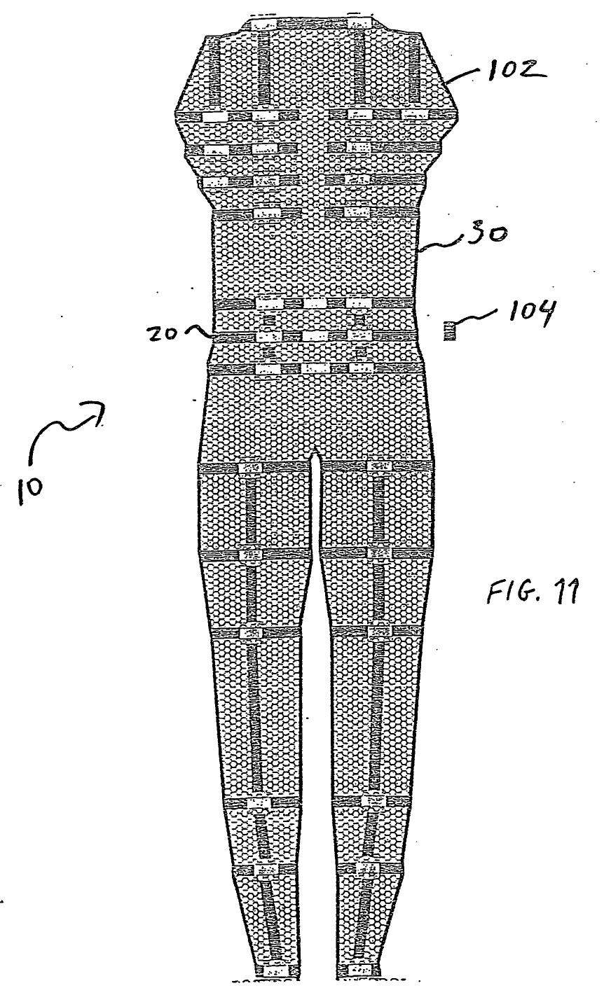

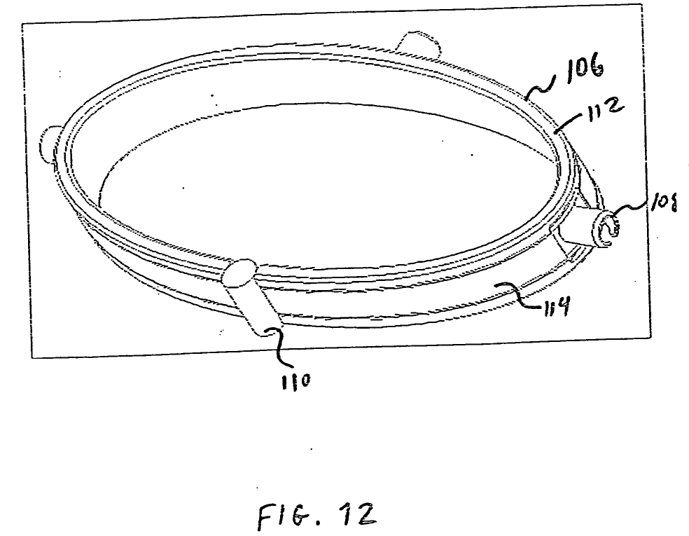

FIG. 9 is a schematic drawing illustrating support segments 22 of adjacent support elements 20 attached to spinal fasteners 54. At the site of attachment of a support segment 22 to a spinal fastener 54 there is a hinge of rotation 92. This is adjustable to allow varying degrees of rotational movement at this site of attachment. In certain embodiments of the device, running through vertically adjacent spinal fasteners and other vertically adjacent components of backbone 60 is at least one axial spanning component 50. Axial spanning components 50 conferring varying degrees of rigidity and flexibility run vertically and connect adjacent spinal fasteners and support elements. Axial spanning components 50 are preferably here cables that can be tightened and loosened in real time to adjust a degree of flexibility and rigidity. Axial spanning components 50 can be designed in short segments so that individual levels as well as individual sides (only left, right or center), for example, can be fixed while allowing other levels and sides mobility. Axial spanning components 50 contain a mechanism for maintaining the degree of tension (tightening) such as a locking mechanism that maintains the length and tension of the axial spanning component 50.

An important feature of the device of the present invention is that support elements 20 are also of varying and adjustable rigidity and tension. The design and construction of support elements 20 and the component support segments 22 that constitute support elements 20 is such as to allow this variation and adjustment in real time. For example, support segments 22 can be constructed as tubes which vary in elasticity based on a degree of inflation of the tubes. Tubes are sealed so as to form a bladder. Partially inflated tubes are more elastic while fully inflated tubes are rigid. The tubes can be inflated with a gas such as compressed air or a liquid such as water. The tubes in certain configurations have such components as inflation valves for attachment to devices such as an air compressor or other source of pressured air, gas or other suitable fluid to vary the degree of inflation (pressure) and thus the tension of the tube which varies the rigidity of the support element 20. Such configurations also contain devices for measuring and adjusting the pressure within the tube, which can be under the control of a pro able control mechanism. In an alternate configuration, support segments 22 constitute an outer shell with an inelastic and adjustable component within the shell such as at least one cable 90. In certain configurations, such as that illustrated in FIG. 10 the outer shell of support segment 22 is composed of a plurality of individual subsegments 96. The individual subsegments 96 as well as tube and the shell described hereinabove are made of any flexible but strong material which generally has minimal elasticity such as fabric, nylon, canvas, plastic and so forth, preferably a material such as latex. Cable 90 that can be tightened and loosened in real time to adjust a degree of flexibility and rigidity. Cable 90 has a locking mechanism that maintains the desired amount of tension in cable 90 and thus in support element 20. In certain embodiments, the degree of tightening (tension) including the locking of cable 90 and of axial spanning component 50 is under the control of a tension control mechanism which may be adjustable and programmable cables and fasteners are generally constructed from strong and durable materials such as metals, alloys, plastics and the like.

FIG. 10 illustrates a still further preferred embodiment of the fastening of a support element 20 around a region of the body, using a lock fastener 100. At one end of support element 20 is connected lock body 94. At the other end of support element 20 is connected connecting probe 98. Probe 98 fits into spring loaded lock body 94, which engages probe 98 and holds it locked into place. The spring of lock body 94 must be manually released, disengaging probe 98 to unfasten lock fastener 100.

The point of attachment of axial spanning elements to the support elements is adjustable according to the particular circumstances of treatment. The axial spanning elements are attachable to more than two support elements, and more than two support elements can be attached to one another and to an axial spanning element. The tension adjustment is made in real time, is variable and is adjustable, and is programmable where circumstances require. The variable tension in various support and spanning elements can also be employed to function as an external, artificial muscle. Vertical spinal support (a “backbone”) capable of being adjusted to modify the force and direction of support is provided, of both the entire spine as well as of individual regions. Full rigid support of the entire body is possible. The device uses multiple supports at each region with multiple fixing elements allowing a maximal degree of variability in the directions and specificity of support and manipulation available. The creation of support points at multiple different levels within a single region (e.g. trunk, chest and spine, or forearm) is possible. The elements are enclosed in a garment (suit) making application simple. There is extensive provision for attachment for additional control units and other devices such as wheels and driving mechanisms to actively move and ambulate the patient.

At various points on suit 30 of device 10 various variable tension producing elements can be attached to produce an external artificial duplicate version of the patient's muscles and associated structures (e.g., ligaments). This can be attached to motor assemblies and the like to generate various movements.

In a preferred embodiment of orthotic device 10 of the present invention, suit 30 has enclosed within suit 30 a belt 106. In certain configurations, belt 106 is a specific preferred embodiment of a support element 20. As illustrated in FIG. 12, belt 106 has, in certain configurations, a belt fastener 110 to render it fully circular. Preferably belt fastener 110 is placed at the front of belt 106. Belt 106 is intended for connection of suit 30 to an additional component element used for external support for standing, sitting, or walking etc. Such a support component is equivalent to standing rod 70 as illustrated in FIG. 8, but of greater complexity. Belt 106 itself confers a degree of rigidity and support to the region belt 106 encloses as well. The belt generally encloses a region in the area of the waist, lumbar region, pelvis and hips. The height of the belt is selected for the desired purpose and in some configurations is adjustable in height as well as position within suit 30 and thus upon the trunk of the patient to which it is applied. Belt 106 is constructed from a material of variable elasticity chosen specifically for the degree of elasticity or rigidity desired. Elements of additional rigidity can be introduced within (112) or upon (114) belt 106 as indicated. The adjustability of flexibility/rigidity of belt 106 is in certain configurations similar to that for other support elements 20 as described hereinabove. (Throughout the specification and the accompanying claims there is an inverse relationship between flexibility and rigidity, from which one can be deduced from the other.)

Belt 106 has at least one belt connector 108 which is used to connect belt 106 to the additional support component. Preferably a plurality of belt connectors 108 are employed; three are illustrated in FIG. 12, at the back and lateral margins of belt 106.

As seen in FIG. 13, connector 108 on belt 106 preferably takes the form of a socket into which a ball component 120 on the additional support component 122 (illustrated as an example in FIG. 14) can be inserted for attachment of belt 106 to additional support component 122. A specifically shaped cut-out 116 in wall 118 of connector 108 allows ball component 120 to be inserted into the center socket 114 of connector 108. Thus the connection made between suit 30 and additional support component 122 is via at least one ball and socket joint (124) at the region of the pelvis and hip via belt 106. Such an arrangement approximates the normal anatomic functioning of the hip joint FIG. 14 shows ball 120 being placed in approximation to socket 114 prior to being inserted therein. Stalk 126 leading to ball 120 is in some configurations a spring to confer further freedom of movement. The tension and rigidity of the spring of stalk 126 may be variable to specifically allow particular desired degrees of flexibility.

FIG. 14 further schematically illustrates joint 124. Ball 120 is attached via stalk 126 to ball joint base 128, which is itself attached to vertical rod 136 via plate 132, to which base 128 is connected by at least one adjustable tightener 130. Base 128 has a central axle 134 which traverses a channel 138 (see FIG. 14) in plate 132 and rod 136. Axle 134 holds the ball assembly 140 generally in place in relation to rod 136, but it is the at least one tightener 130 which fastens base 128 to rod 136. The at least one tightener 130 which fastens base 128 to rod 136 is of adjustable tension, conferring a variable degree of freedom of movement to joint 124. Depending on the number and placement and alignment of the at least one tightener 130 and which of these are adjusted to what degree of tension determines the precise axis of movement and its degree of freedom in that axis. Tightener 130 is preferably an adjustable screw. In a preferred embodiment there are at least 3, and preferably 4, tighteners 130 at each joint 124.

As illustrated in FIG. 14, in some preferred embodiments, plate 132 is attached, not directly to vertical rod 136, but rather to a horizontal bar 142, horizontal bar 142 further being fixed to vertical rod 136. The various rods and bars of the present invention are generally fabricated form suitable materials such as metals including, but not limited to steel and aluminum. Other components such as fasteners, rollers, springs, cables and the like are fabricated from suitable and appropriate materials including metals and plastics as is standard in the art. Such an embodiment establishes a more natural pelvis-like configuration. As illustrated in FIG. 14, vertical rod 136 is further attached to a standing support bar 144. The attachment of rod 136 to bar 144 is via a similar attachment as that which attaches rod 136 to base 128. Preferably there is a support bar attachment axle 146 which generally holds bar 144 in place relative to rod 136 through passage through a channel 146 on rod 136, and at least one tightening screw 148 which firmly, but adjustably and variably, holds bar 144 in place. As may be seen in FIGS. 14 and 16, preferably device 10 has 2 rods 136 and 2 standing support bars 144.

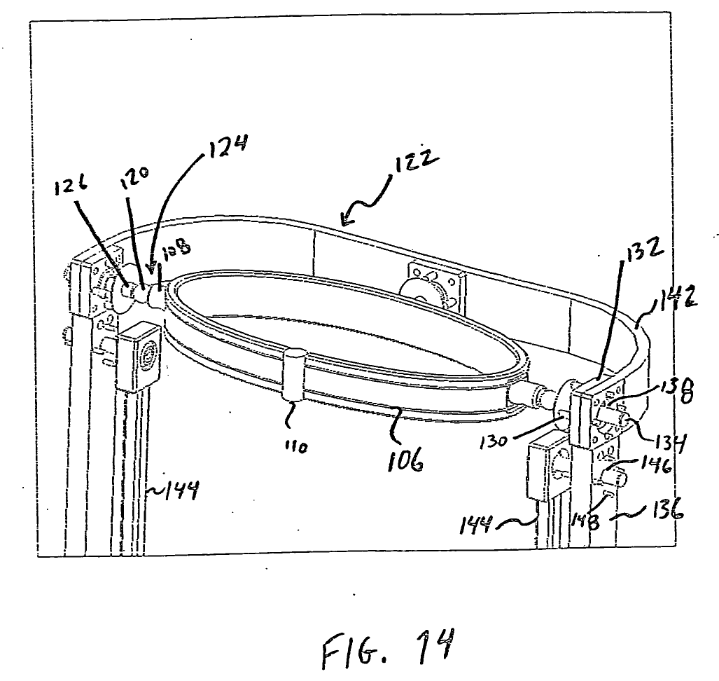

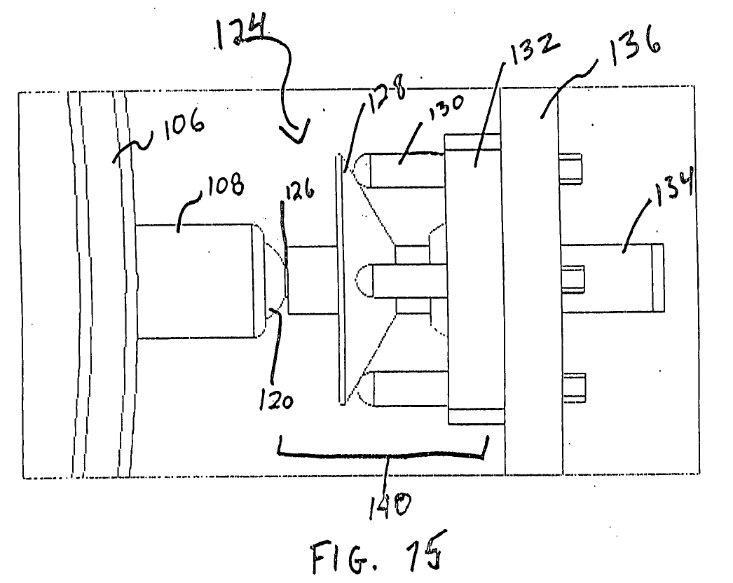

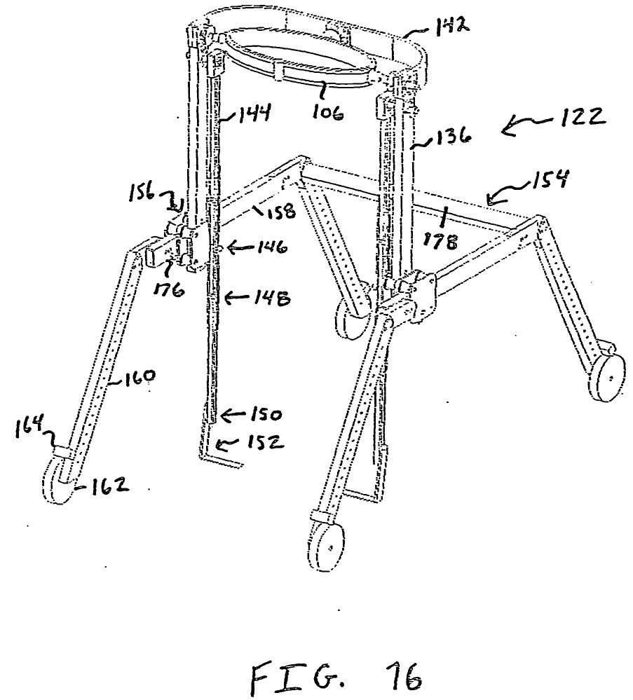

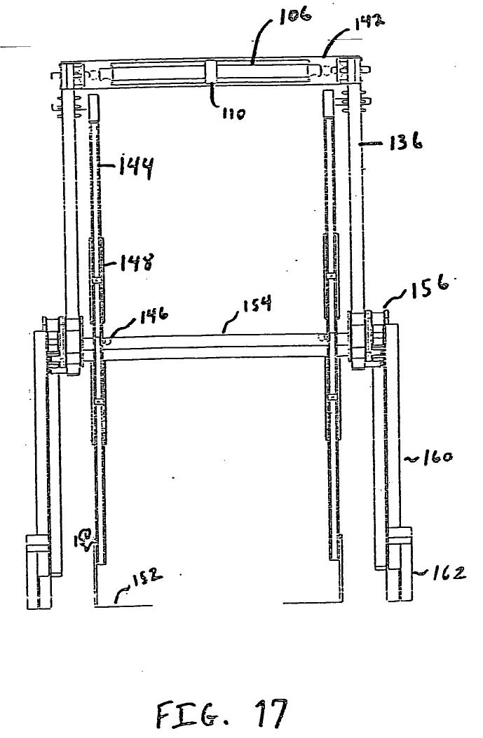

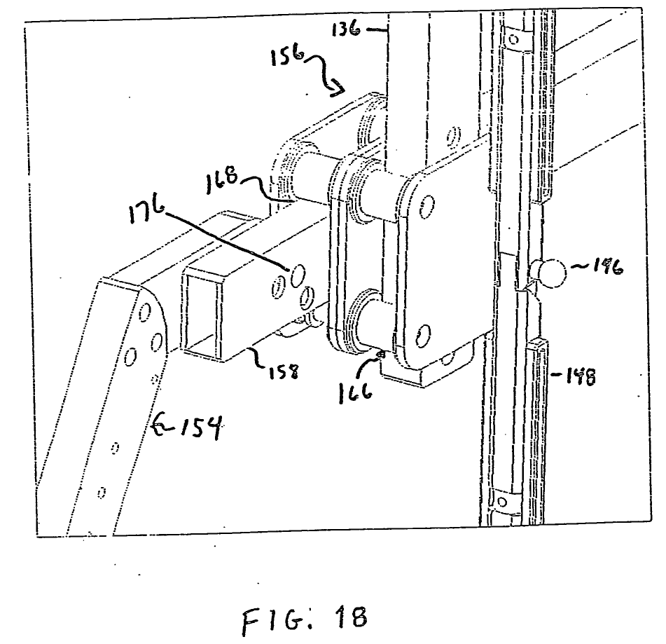

The at least one standing support bar 144 that is part of device 10 is located then outside of suit 30. Standing support bar 144 is located outside of the body of the patient and in some configurations is fastened separately to at least one point on at least one lower limb of a patient, with or without the limb being enclosed in suit 30, while in other preferred embodiments is not fastened to a limb of the patient. Points of fastening are preferably in segments of the limb between joints. In certain preferred embodiments, standing support bar 144 contains at least one joint corresponding to a position of an anatomical joint on the lower extremity of the patient. In FIG. 16, two such joints are illustrated on each standing support bar 144, and are indicated as 146 and 150. Joints 146 and 150 are preferably variably adjustable hinges that can be adjusted to remain rigidly fixed or to allow various amounts of motion in at least one axis of movement. In addition in certain preferred embodiments, standing support bar 144 is equipped with a length adjustment mechanism 148 for variably adjusting the length of standing support bar 144. Such adjustment allows for device 10 to be used in patents of different sizes and heights, to allow for device 10 to be used in the same patient as he changes in height over time or to allow for support in different positions, such as sitting and standing, as non-limiting examples. In certain preferred embodiments at the distal end of standing support bar 144 is located a ground support element 152 offering a larger surface area element to come into contact with the surface on which the patient is supported. Ground support element 152 may further be attached to the foot or footwear of the patient. These elements can also be seen in the frontal view illustrated in FIG. 17.

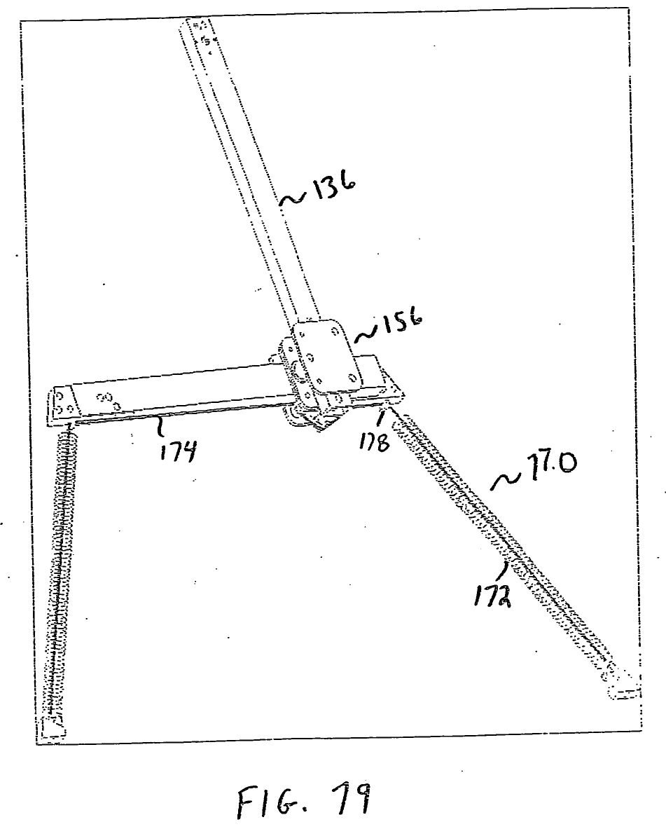

Vertical rod 136, which is attached at the proximal or upper end to belt 106 either directly or via horizontal bar 142 using the ball and socket joint, is attached at the lower or distal end to walker frame 154 via a specialized frame connection 156. Specialized frame connection 156 is illustrated in FIGS. 16-19. As illustrated in FIG. 18, vertical rod 136 passes through vertical channel 166 and horizontal frame element 158 of walker frame 154 passes through horizontal channel 168 such that the position of vertical rod 136 relative to walker frame 154 may be variably adjusted both in a vertical, up-down dimension as well as in a horizontal, front-back axis. A variably adjustable fixation mechanism, such as at least one screw, knob, rollers, thumbscrew, friction mechanism, or other such suitable mechanism for maintaining the elements relative positions to one another that can be variably adjusted is preferably employed in specialized frame connection 156. These adjustments allow device 10 to be used in various positions—for example, standing, sitting, and squatting, and to adjust for the point of the center of gravity to improve balance.

Walker frame 154 further includes at least one wheel support 160 attached to at least one horizontal frame element 158 as illustrated in FIG. 16. Wheel support connection 176 can be loosened or tightened to allow folding of walker frame 154 for easy portability as discussed further hereinunder. For stability and for ease of locomotion additional support component 122 preferably includes a plurality of wheel supports 160 each attached to a wheel 162. In various configurations, preferred embodiments of the present invention employ 2, 3 or 4 wheel supports 160 each attached to a wheel 162. Other connecting rods, such as is 178 illustrated in FIG. 16 are employed depending on the number of wheel supports and exact configurations. At least one wheel 162 is further provide with a brake mechanism 164. In this manner additional support component 122 allows the patient to stand supported and as well to locomote, as with a walker.

Device 10 supports the patient in a vertical position, allows prolonged standing, allows walking with hands free and can support, correct or stimulate muscular movement and coordination along various axes of movement. The support afforded by additional support component 122 regulates a reduction or increase in gravitational loading. Position changes are permitted and facilitated. Varying degrees of support, for example, by fastening to only one lower limb, and allowing standing on one limb, can be offered. Positions such as squatting can be permitted and even facilitated. This can be employed in exercise regimens and physiotherapy. The adjustable connections and joints permit regulation of the axes of movement and degree of freedom permitted or required. This promotes rehabilitation and can help maintain and improve balance and support. The range of exercise that can be performed is larger than with prior art orthotic devices. Adjustments are possible in a maximal number of planes, each independently or together as indicated. Weights, tension elements such as springs or other devices can be added for physiotherapy and rehabilitation purposes.

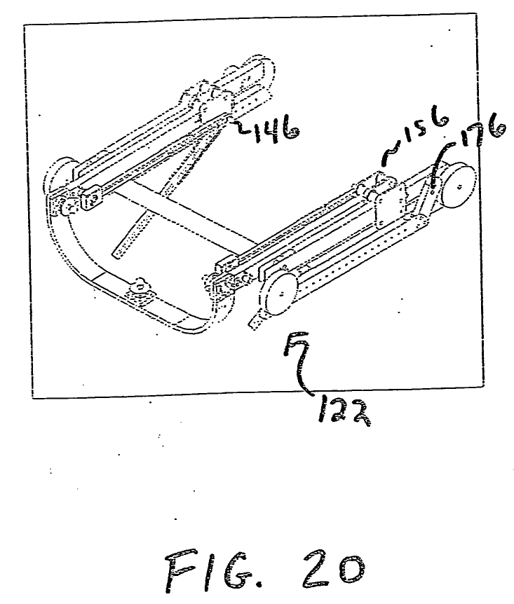

Cut away schematic diagram FIG. 19, illustrates one example of the use of a spring tension configuration in a preferred embodiment of the present invention. Spring tension unit 170 within wheel support 160 of walker frame 154 provides counter tension when the weight, center of balance and support are shifted during a patient's position change as with moves between standing and sitting. Spring tension unit 170 employs for example, at least one spring 172 connected to the distal end of at least one wheel support 160 and a cable 174, to which spring 172 is attached to at at least one end, as well as at least one cable roller 178. When sitting, for example, spring tension unit 170 can apply brake 164 to stop the patient from rolling away while moving into the sitting position. When standing, spring tension unit 170 can be configured to impel the patient upward.

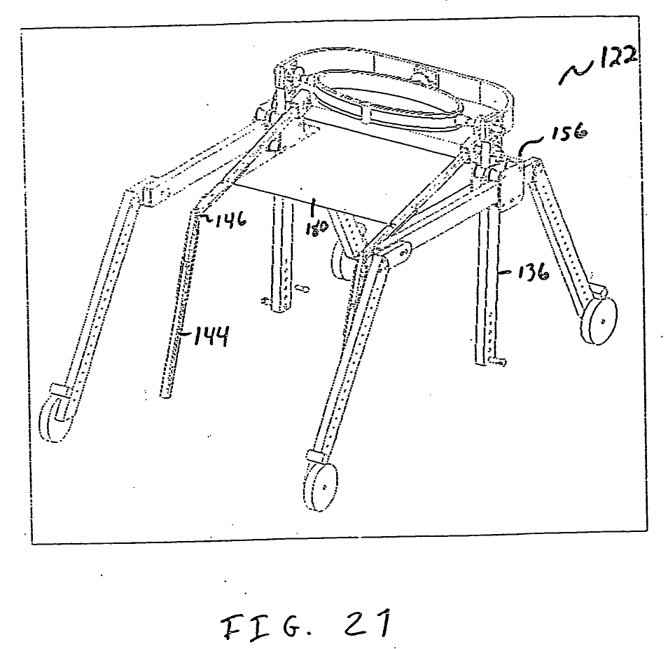

FIG. 20 illustrates that additional support component 122 can be easily folded at various connections and joins such as 146, 156, and 176 permitting it to be folded into a compact shape and size allowing for easy stowage and convenient portability, improving its functionality.

FIG. 21 illustrates a further preferred embodiment in which a seat 180 is further incorporated into additional support component 122. Seat 180 allows further relief of a gravitational load and allows the person to sit from a standing position for variable amounts of time. Seat 180 incorporates such additional components as hinges, connections, weight supports, springs and the like to enable seat 180 to be folded out of place when not in use and to support the patient for prolonged periods of time when the patient sits thereupon. FIG. 21 further illustrates that vertical rod 136 can be adjusted at connection 156, and standing support bar 144 at joint 146, to allow sitting. Additional support component 122 can thus be converted to use as a wheelchair type device from a walker, as well as vice verse.

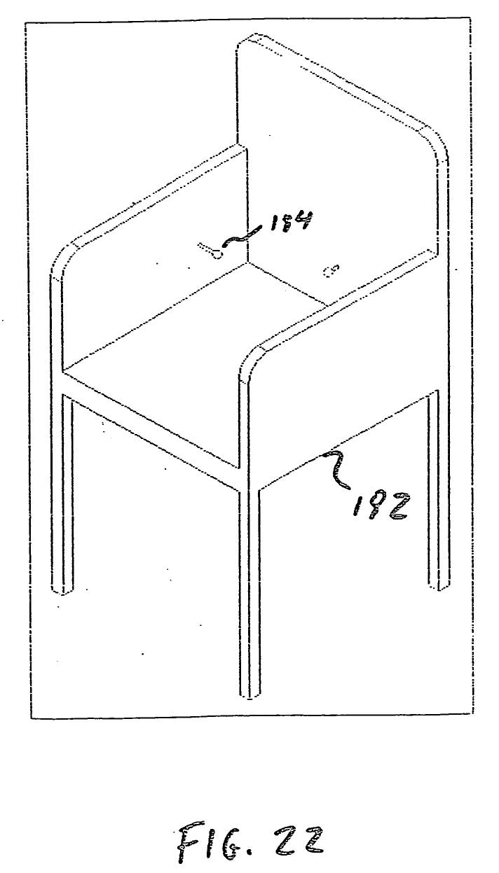

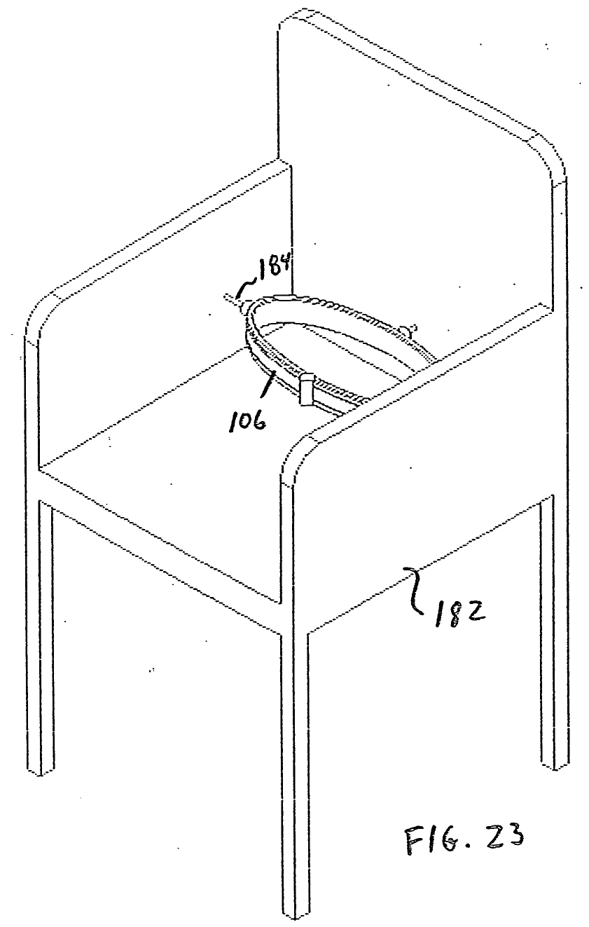

For more long term, fixed positioning in a seated position an alternative chair device 182 (as illustrated in FIG. 22) can be employed rather than additional support component 122. As a non-limiting example, chair 182 can include at least one ball type connector 184 which can attach to a socket connector 108 on belt 106 within suit 30, as illustrated in FIG. 23.

One of ordinary skills in the art would know how to operatively assemble these components. The particular components shown are not necessarily exhaustive of the components contained therein, which any other components are generally known in the art. For example, additional components may be required for making connections. Elements not specifically shown or described herein may be selected from those known in the art. The present invention further contemplates substitutes of the components shown that are capable of carrying out substantially the same functions.

Device 10 thus utilizes spinal support to help maintain vertical posture. However whereas rigid fixation of the spine typically results in significant impairment of biomechanics of movement, employment of device 10 with devices such as additional support component 122 allows freedom of movement, and allows normal biomechanics of the musculoskeletal system, and normal performance of stereotyped movements, such as walking. This is further increased due to features such as the individualized support of specific segments of the trunk with the degree of rigidity, its axis and level all being customizable. For example, as opposed to other prior art inventions, there exists in the present invention an external analogue of the hip joint. Whereas fixation in prior art devices results in extinguishing of reflexes and muscular atrophy of the musculoskeletal components of straightening or balance these are preserved in the device of the present invention. Device 10 can be used both passively and actively—for structural support only or to actively enhance movement or even fully produce it actively. Compared to prior art devices, the device of the present invention can be adjusted in height and level of support, for example, of the torso, allowing adaptation to a growing patient, and developmental adaptations. It can be used for prolonged standing, in a sitting position, and allows movement from standing to sitting. It is foldable and portable, can be donned and attached, as well as adjusted or removed easily. Device 10 can further be used for the treatment of problems such as scoliosis.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. A customizable orthotic bracing device with adjustable support and forces, the device to be applied to the body of an individual, for treatment and rehabilitation of disorders of the musculoskeletal and nervous system, the device comprising:
a. a wearable garment, and
b. a plurality of support elements, said support elements being of variable and adjustable flexibility, said support elements being connected to said wearable garment.

2. The orthotic bracing device of claim 1, further including
c. at least one axial spanning element, said at least one axial spanning element being of variable and adjustable flexibility, said at least one axial spanning element being connected to at least two of said support elements.

3. The orthotic bracing device of claim 1, wherein said support elements are contained within layers of said garment.

4. The orthotic bracing device of claim 1, wherein said support elements are located on the outer surface of said garment.

5. The orthotic bracing device of claim 1, wherein said garment covers substantially the entire body of the individual.

6. The orthotic bracing device of claim 1, wherein said garment is composed of a plurality of individual compartments.

7. The orthotic bracing device of claim 6, said compartments enclosing a plurality of spaces, wherein at least one insert is inserted into at least one of said spaces.

8. The orthotic bracing device of claim 7, wherein said at least one insert is rigid.

9. The orthotic bracing device of claim 7, wherein said at least one insert is buoyant.

10. The orthotic bracing device of claim 7, wherein said at least one insert is fabricated from a polystyrene foam.

11. The orthotic bracing device of claim 1, wherein said garment is fabricated of a waterproof material.

12. The orthotic bracing device of claim 1, wherein said device is adapted for use in hydrotherapy.

13. The orthotic bracing device of claim 1, wherein said plurality of support elements are arranged in a parallel configuration on at least one region of the body of the individual.

14. The orthotic bracing device of claim 1, wherein at least one of said support elements is constructed from a plurality of support segments.

15. The orthotic bracing device of claim 1, wherein said support elements are arranged horizontally.

16. The orthotic bracing device of claim 1, wherein at least one of said support elements is connected to a sternal fastener for providing additional rigidity analogous to a sternal bone of the individual.

17. The orthotic bracing device of claim 1, wherein at least one of said support elements is connected to a spinal fastener for providing additional rigidity analogous to a vertebral bone of the individual.

18. The orthotic bracing device of claim 17, comprising a plurality of spinal fasteners.

19. The orthotic bracing device of claim 18, wherein a plurality of parallel support elements, each of said support elements being connected to a spinal fastener, are axially connected in a posterior portion of the device so as to constitute a backbone.

20. The orthotic bracing device of claim 18, wherein at least two adjacent of said spinal fasteners are connected by at least one tension connector.

21. The orthotic bracing device of claim 20, wherein said flexibility of said at least one tension connector is variable.

22. The orthotic bracing device of claim 20, wherein said flexibility of said at least one tension connector is adjustable.

23. The orthotic bracing device of claim 1, further including at least one moveable joint connection functionally connected to at least one support element.

24. The orthotic bracing device of claim 1, further including at least one moveable joint connection functionally connected to at least one axial spanning element.

25. The orthotic bracing device of claim 1, further including a positional support element for assisting the individual to maintain a desired posture, said standing support element functionally connected to at least one of said support elements.

26. The orthotic bracing device of claim 25, wherein said desired posture is standing.

27. The orthotic bracing device of claim 25, wherein said desired posture is sitting.

28. The orthotic bracing device of claim 25, wherein said positional support element is connected to said at least one of said support elements by at least one ball and socket joint connection.

29. The orthotic bracing device of claim 28, wherein at least one ball of said at least one ball and socket joint is a component of said positional support element.

30. The orthotic bracing device of claim 29, wherein said at least one ball is connected to said positional support element by at least one element conferring a variable degree of freedom of movement.

31. The orthotic bracing device of claim 29, wherein said at least one ball is connected to said positional support element by at least one element conferring an adjustable degree of freedom of movement.

32. The orthotic bracing device of claim 25, wherein said positional support element includes a standing support bar functionally connected to said at least one support element.

33. The orthotic bracing device of claim 32, wherein said standing support bar is functionally connected to said at least one support element by a movable joint connection.

34. The orthotic bracing device of claim 32, wherein said standing support bar is of adjustable length.

35. The orthotic bracing device of claim 32, wherein said standing support bar includes at least one hinge joint along the length of said standing support bar.

36. The orthotic bracing device of claim 25, wherein said positional support element includes a walker frame, said walker frame functionally connected to said support element by a vertical rod.

37. The orthotic bracing device of claim 36, wherein said walker frame is connected to said vertical rod by a frame connection allowing adjustment of a position of said vertical rod vertically and in a forward and backward direction relative to said walker frame.

38. The orthotic bracing device of claim 36, wherein said walker frame is further connected to at least one wheel.

39. The orthotic bracing device of claim 38, wherein said walker frame is further connected to two wheels.

40. The orthotic bracing device of claim 38, wherein said walker frame is further connected to three wheels.

41. The orthotic bracing device of claim 38, wherein said walker frame is further connected to four wheels.

42. The orthotic bracing device of claim 38, further including a brake mechanism attached to the at least one wheel.

43. The orthotic bracing device of claim 25, wherein said positional support element is adapted so as to be foldable.

44. The orthotic bracing device of claim 25, wherein said positional support element includes a seat.

45. The orthotic bracing device of claim 25, wherein said positional support element is adapted so as to allow the patient to move between standing and sitting positions.

46. The orthotic bracing device of claim 1, further including a motion element for assisting the individual in moving at least one part of the body, said motion element functionally connected to at least one of said support elements.

47. The orthotic bracing device of claim 46, further including a motor assembly functionally connected to at least one of said support elements for actively moving at least one portion of the body.

48. The orthotic bracing device of claim 1, wherein said axial spanning element is a cable, adapted so as to capable of being tension adjustable, so as to adjust a degree of flexibility of said cable.

49. The orthotic bracing device of claim 1, wherein said support elements are inflatable tubes.

50. The orthotic bracing device of claim 1, wherein at least one of said support elements include a cable, adapted so as to capable of being tension adjustable, so as to adjust a degree of flexibility of said cable, thereby adjusting the flexibility of said support element.

51. The orthotic bracing device of claim 1, wherein the device is adapted for use in treatment of cerebral palsy.

52. The orthotic bracing device of claim 1, wherein the device is adapted for use in treatment of scoliosis.
